(19) United States
(12) Patent Application Publication
(10) Pub. No.: US 2024/0109846 A1
(43) Pub. Date: Apr. 4, 2024

(54) PROCESSES RELATED TO FORMATION OF N-(3-CHLORO-1-(PYRIDIN-3-YL)-1H-PYRAZOL-4-YL)-2-(METHYLSULFONYL)PROPANAMIDE

(71) Applicant: CORTEVA AGRISCIENCE LLC, INDIANAPOLIS, IN (US)

(72) Inventors: CARL VINCENT DEAMICIS, INDIANAPOLIS, IN (US); ELIZABETH O. MCCUSKER, CARMEL, IN (US); RAFAEL SHAPIRO, WILMINGTON, DE (US); QIANG YANG, ZIONSVILLE, IN (US)

(73) Assignee: CORTEVA AGRISCIENCE LLC, INDIANAPOLIS, IN (US)

(21) Appl. No.: 18/262,233

(22) PCT Filed: Jan. 20, 2022
(86) PCT No.: PCT/US22/13039

§ 371 Date: Jul. 20, 2023

Related U.S. Application Data
(60) Provisional application No. 63/139,882, filed on Jan. 21, 2021.

(51) Int. Cl. C07D 231/40; C07D 401/04

(52) U.S. Cl. CPC C07D 231/40; C07D 401/04

(57) ABSTRACT

This disclosure relates to a molecule, N-(3-chloro-1H-pyrazol-4-yl)-2-(methylsulfonyl) propanamide (S2b)

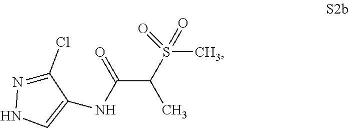

and processes to prepare S2b and N-(3-chloro-1-(pyridin-3-yl)-1H-pyrazol-4-yl)-2-(methylsulfonyl) propanamide (S3b)

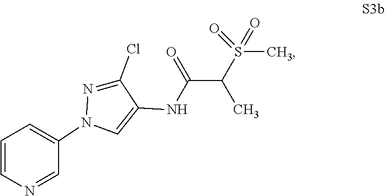 [SMILES: CC(C(=O)Nc1cn(-c2cccnc2)nc1Cl)S(C)(=O)=O]

having pesticidal utility against pests in Phyla Arthropoda, Mollusca, and Nematoda.

### CROSS REFERENCE TO RELATED TO APPLICATIONS

This application claims priority to International (PCT) Patent Application Serial No. PCT/US2022/013039, filed Jan. 20, 2022, and entitled “PROCESSES RELATED TO FORMATION OF N-(3-CHLORO-1-(PYRIDIN-3-YL)-1H-PYRAZOL-4-YL)-2-(METHYLSULFONYL) PROPANAMIDE”, which claims priority to and the benefit of U.S. Provisional Patent Application Ser. No. 63/139882 filed Jan. 21, 2021, the complete disclosures of which are expressly incorporated by reference herein.

### BACKGROUND

This disclosure relates to processes to prepare N-(3-chloro-1-(pyridin-3-yl)-1H-pyrazol-4-yl)-2-(methylsulfonyl) propanamide having pesticidal utility against pests in Phyla Arthropoda, Mollusca, and Nematoda.

1-(Pyridin-3-yl)-1H-pyrazoles have been disclosed in application WO2019/236274.

The protection of crops from insects and nematodes which inhibit crop growth is a constantly recurring problem in agriculture. To help combat this problem, researchers in the field of synthetic chemistry have produced an extensive variety of chemicals and chemical formulations effective in the control of such insects and nematodes. Chemical insecticides and nematicides of many types have been disclosed in the literature and a large number are in commercial use. However, there remains a need for compositions and methods that are effective in controlling undesirable insects and nematodes and methods to prepare such.

### SUMMARY

A molecule, N-(3-chloro-1H-pyrazol-4-yl)-2-(methylsulfonyl)propanamide (S2b), having the following formula

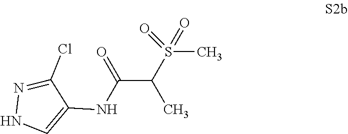

is provided. Additionally, processes to make and use a molecule of formula S2b are provided.

### DEFINITIONS

Examples provided herein are not exhaustive and should not be construed as limiting. It is understood that a substituent should comply with chemical bonding rules and steric compatibility constraints in relation to the particular molecule to which it is attached. These definitions are only to be used for the purposes of this disclosure.

The term “alkyl” means an acyclic, saturated, branched, or unbranched, substituent consisting of carbon and hydrogen, for example, methyl, ethyl, propyl, isopropyl, butyl, sec-butyl, isobutyl, and tert-butyl.

The term “halogen” or “halo” or derivative terms such as “halide” refers to one or more halogen atoms, defined as F, Cl, Br, and I.

The term “ambient pressure” refers to pressures from about 80 kilopascals (kPa) to about 105 kPa.

The term “ambient temperature” or “room temperature” refers to temperatures ranging from about 20° C. to about 24° C.

The term “catalyst” refers to any substance that increases the rate of a reaction without itself being consumed.

“Continuous flow”, “flow”, “continuous formation”, “continuous process”, or other derivative terms as used herein means methods that produce a minimum amount of a reactive intermediate at any given time and provide reduced cycle times in comparison to conventional methods. For example, U.S. Pat. No. 9,145,428 B2 describes methods and systems using continuous flow.

All references, including publications, patent applications, and patents, referred to herein are incorporated by reference herein to the same extent as if each reference were individually and specifically indicated to be incorporated by reference and were set forth in its entirety.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DETAILED DESCRIPTION

A molecule, N-(3-chloro-1H-pyrazol-4-yl)-2-(methylsulfonyl)propanamide (also known as “S2b” herein), having the following formula

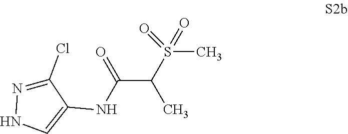

is provided. Additionally, processes to make and use a molecule of formula S2b are provided. The molecule S2b may be useful in the process to prepare N-(3-chloro-1-(pyridin-3-yl)-1H-pyrazol-4-yl)-2-(methylsulfonyl) propanamide (also known as “S3b” herein) and shown below,

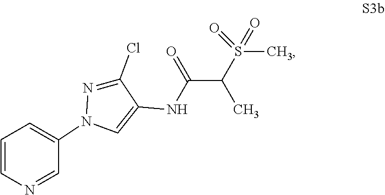 [SMILES: CC(C(=O)Nc1cn(-c2cccnc2)nc1Cl)S(C)(=O)=O]

having pesticidal utility against pests in Phyla Arthropoda, Mollusca, and Nematoda.

The following are processes related to the preparation of S2b and S3b.

A process for the preparation of an activated carboxylic acid S1b is shown in Scheme One. Activated carboxylic acids S1b, wherein A is an activating group, may be an acid halide, such as an acid chloride S1b-1, an acid bromide, or an acid fluoride; a mixed anhydride S1b-2; an acyl carbonate S1b-3; or an ester S1b-4, such as a methyl ester, an ethyl ester, or a propyl ester. The conversion of a carboxylic acid S1a to an activated carboxylic acid S1b shown in Scheme One is conducted in the presence of a carboxylic acid activator.

### Scheme One

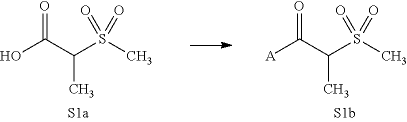

wherein A is Cl (S1b-1), O(C═O)R₁ (S1b-2), O(C═O)OR₁ (S1b-3), or OR₁ (S1b-4), wherein R₁ is (C₁-C₄)alkyl.

Acid chlorides S1b-1, wherein A is Cl, may be prepared from the corresponding carboxylic acids, such as 2-(methylsulfonyl)propanoic acid (S1a), by treatment with a carboxylic acid activator such as a dehydrating chlorinating reagent, for example oxalyl chloride or thionyl chloride. Optionally, a catalyst (e.g., an “acid chloride formation catalyst”) may be used to promote the reaction of S1a to the acid chloride S1b-1. Examples of acid chloride formation catalysts include, but are not limited to, N,N-dimethylformamide (“DMF”) and 1-formylpiperidine.

Mixed anhydrides S1b-2, wherein A is O(C═O)R₁, wherein R₁ is (C₁-C₄)alkyl, may be prepared from carboxylic acids, such as 2-(methylsulfonyl)propanoic acid (S1a), with a carboxylic acid activator such as an acid chloride, for example, pivaloyl chloride, or an anhydride such as pivalic anhydride. Optionally, a catalyst (e.g., a “mixed anhydride formation catalyst”) may be used to promote the reaction of S1a to the mixed anhydride S1b-2. Examples of mixed anhydride formation catalysts include, but are not limited to, N,N-dimethyaminopyridine (“DMAP”) and N-methylimidazole (“NMI”).

Acyl carbonates S1b-3, wherein A is O(C═O)OR₁, wherein R₁ is (C₁-C₄)alkyl, may be prepared from the reaction of the corresponding carboxylic acids, such as 2-(methylsulfonyl)propanoic acid (S1a), with a carboxylic acid activator such as a chloroformate (R₁O(C═O)Cl, wherein R₁ is (C₁-C₄)alkyl), such as methyl, ethyl, isobutyl chloroformate, or mixtures thereof. Optionally, a catalyst (e.g., an “acyl carbonate formation catalyst”) may be used to promote the reaction of S1a to the acyl carbonate S1b-3. Examples of acyl carbonate formation catalysts include, but are not limited to, N,N-dimethyaminopyridine (“DMAP”) and N-methylimidazole (“NMI”).

Esters S1b-4, wherein A is OR₁, wherein R₁ is (C₁-C₄)alkyl, can be generated from the reaction of the corresponding carboxylic acids, such as 2-(methylsulfonyl)propanoic acid (S1a), with alcohols such as methanol, ethanol, and propanol under acidic conditions, or with other methods known in the art, for example, by using coupling reagents.

In general, about 1.0 moles to about 5 moles of activator per mole of S1a, more preferably, about 1.0 moles to about 1.5 moles of activator per mole of S1a may be used.

The reaction in Scheme One is conducted in the presence of an aprotic solvent. Examples of aprotic solvents are heptanes, chloroform (“CHCl₃”), 1,2-dichloroethane (“DCE”), toluene (“PhCH₃”), ethyl acetate (“EtOAc”), tetrahydrofuran (“THF”), 2-methyltetrahydrofuran (“2-MeTHF”), methyl tert-butyl ether (“MTBE”), cyclopentyl methyl ether (“CPME”), dichloromethane (“DCM”), and acetonitrile (“ACN”). Optionally, mixtures of such solvents may be used. Alternatively, the reaction in Scheme One may be conducted without solvent.

The reaction in Scheme One may be conducted at ambient temperatures and ambient pressures. However, higher, or lower temperatures and pressures may be used. For the embodiments provided herewith, temperatures from about −10° C. to about 110° C. may be used, preferably temperatures from about −10° C. to about 90° C. may be used.

The molecule S1b may be isolated and used or used without isolation from the solvent in a continuous manner in the subsequent reaction.

A process for the preparation of N-(3-chloro-1H-pyrazol-4-yl)-2-(methylsulfonyl) propanamide (S2b) is shown in Scheme Two. 3-Chloro-1H-pyrazol-4-amine hydrochloride (S2a) is reacted with an activated carboxylic acid S1b to afford the molecule S2b.

### Scheme Two

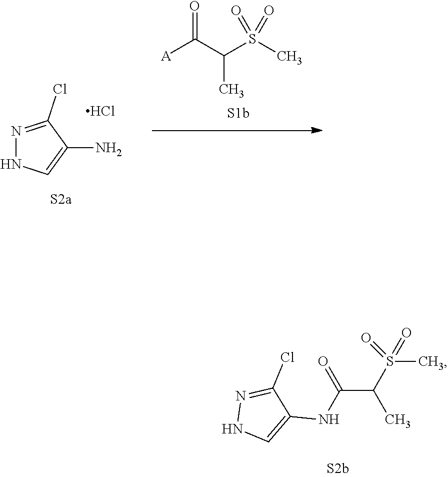

wherein A is Cl, O(C═O)R₁, O(C═O)OR₁, or OR₁, wherein R₁ is (C₁-C₄)alkyl.

The reaction in Scheme Two is conducted in the presence of a base. Examples of bases are organic bases and inorganic bases. Examples of organic bases are pyridine, lutidine, 2-picoline, N,N-diisopropylethylamine (“DIPEA”) and triethylamine (“TEA”). Examples of inorganic bases are sodium hydroxide (“NaOH”), potassium hydroxide (“KOH”), potassium carbonate (“K₂CO₃”), potassium bicarbonate (“KHCO₃”), sodium carbonate (“Na₂CO₃”), and sodium bicarbonate (“NaHCO₃”). In general, about 1 mole to about 5 moles of base per mole of S2a can be used; more preferably, about 2.0 moles to about 3.5 moles of base per mole of S2a may be used. Optionally, a catalyst (e.g., an “amide bond formation catalyst”) may be used to promote the reaction of S2a to S2b. Examples of amide bond formation catalysts include, but are not limited to, N,N-dimethyaminopyridine (“DMAP”) and N-methylimidazole (“NMI”).

The reaction in Scheme Two is conducted in the presence of an aprotic solvent or a protic solvent. Examples of aprotic solvents are heptanes, chloroform (“CHCl₃”), dichloroethane, toluene (“PhCH₃”), dichloromethane (“DCM”), tetrahydrofuran (“THF”), 2-methyltetrahydrofuran (“2-MeTHF”), methyl tert-butyl ether (“MTBE”), cyclopentyl methyl ether (“CPME”), acetonitrile (“ACN”), and ethyl acetate (“EtOAc”). Examples of protic solvents are n-butanol (“n-BuOH”), isopropanol (“i-PrOH”), n-propanol (“n-PrOH”), ethanol (“EtOH”), methanol (“MeOH”), and water (“H₂O”). Mixtures of solvents, such as toluene and water or tetrahydrofuran and water, may be used.

The reaction in Scheme Two may be conducted at ambient temperatures and pressures. However, higher or lower temperatures and pressures may be used. Currently, temperatures from about −10° C. to about 110° C. may be used; preferably temperatures from about −10° C. to 90° C. may be used. The reaction in Scheme Two may be conducted at ambient pressures.

The compound S2b may be isolated and used or used without isolation from the solvent in a continuous manner in the subsequent reaction.

A process for the preparation of N-(3-chloro-1-(pyridin-3-yl)-1H-pyrazol-4-yl)-2-(methylsulfonyl) propanamide (S3b) is shown in Scheme Three. N-(3-Chloro-1H-pyrazol-4-yl)-2-(methylsulfonyl) propanamide (S2b) is reacted with a 3-halopyridine (S3a), wherein X is Br, Cl, or I to afford the molecule S3b.

### Scheme Three

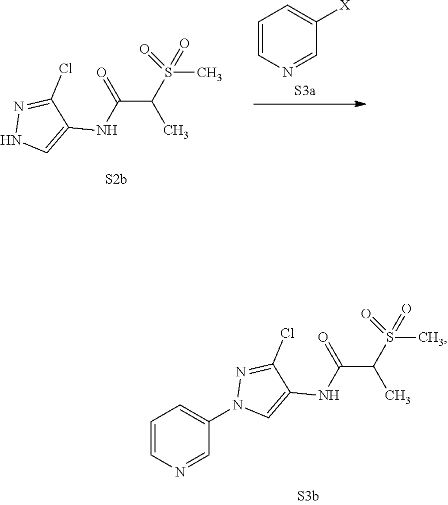

wherein X is Br, Cl, or I.

The reaction in Scheme Three is conducted in the presence of a base. Examples of bases are organic bases and inorganic bases. Examples of organic bases are pyridine, lutidine, 2-picoline, N,N-diisopropylethylamine (“DIPEA”) and triethylamine (“TEA”). Examples of inorganic bases are sodium methoxide (“NaOCH₃”), sodium ethoxide (“NaOCH₂CH₃”), lithium hydroxide (“LiOH”), sodium hydroxide (“NaOH”), potassium hydroxide (“KOH”), cesium hydroxide (“CsOH”), calcium hydroxide (“Ca(OH)₂”), sodium diphosphate (“Na₂HPO₄”), potassium phosphate (“K₃PO₄”), sodium phosphate (“Na₃PO₄”), potassium carbonate (“K₂CO₃”), potassium bicarbonate (“KHCO₃”), calcium carbonate (“CaCO₃”), cesium carbonate (“Cs₂CO₃”), lithium carbonate (“Li₂CO₃”), sodium carbonate (“Na₂CO₃”), and sodium bicarbonate (“NaHCO₃”). In general, about 1 mole to about 5 moles of base per mole of S2b can be used; more preferably, about 2.0 moles to about 3.5 moles of base per mole of S2b may be used.

The reaction in Scheme Three is conducted in the presence of a copper halide. Examples of copper(I) and copper(II) halides are copper(I) chloride (“CuCl”), copper(II) chloride (“CuCl₂”), and copper(I) iodide (“CuI”).

The reaction in Scheme Three is conducted in the presence of a ligand. Examples of ligands are pyridine, alkylpyridine, N,N′-dimethylethylenediamine (“DMEDA”), triethylenetetramine (“TETA”), bis(2-hydroxyethyl) ethylenediamine (“BHEEA”), 1-butylimidazole, 8-hydroxyquinoline, L-proline, 2,2-bipyridyl, 1,10-phenanthroline, and pipecolinic acid.

The reaction in Scheme Three is conducted in the presence of an aprotic solvent. Examples of aprotic solvents are ethyl acetate, dioxane, tetrahydrofuran (“THF”), 2-methyltetrahydrofuran (“2-MeTHF”), 1,2-dimethoxyethane (“DME”), dichloromethane (“DCM”), dimethyl sulfoxide (“DMSO”), N-methylpyrrolidone (“NMP”), N,N-dimethylformamide (“DMF”), propionitrile, benzonitrile, acetonitrile (“ACN”), xylenes, toluene (“PhCH₃”), and water. Optionally, mixtures of such solvents may be used. A sparged solvent is preferred.

The reaction in Scheme Three may be conducted at ambient pressures. Temperatures from about 40° C. to about 150° C. may be used, preferably temperatures from about 60° C. to about 120° C. may be used.

The compound S3b may be isolated by conventional methods known in the art.

The following examples are for illustration purposes and are not to be construed as limiting.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Molecules are given their known names, named according to the naming program within ChemDraw (version 17.1.0.105 (19)). If such a program is unable to name a molecule, such molecule is named using conventional naming rules. ¹H NMR spectral data are in ppm (δ) and were recorded at 400 MHz, and ¹³C NMR spectral data are in ppm (δ) and were recorded at 101 MHz, unless otherwise stated.

### EXAMPLE 1

Preparation of 2-(methylsulfonyl) propanoyl chloride (S1b-1)
Step 1a: Preparation of 2-(methylthio)propanoic acid

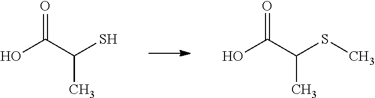

A 2-liter (L) round bottom flask equipped with an overhead stirrer, a temperature probe, and a reflux condenser was charged sequentially with thiolactic acid (139.5 grams (g), 1.32 moles (mol)), dimethyl carbonate (261 g, 2.89 mol), N,N-dimethylformamide (DMF; 0.63 L), and potassium carbonate (109 g, 0.789 mol). The resulting white suspension was heated at an internal temperature of 100° C. for 6 hours (h) and at 50° C. overnight. The white suspension was cooled to room temperature; water (500 milliliters (mL)) was added; and the yellow solution was acidified to pH˜3 with concentrated hydrochloric acid (conc. HCl). The solution was extracted with methyl tert-butyl ether (MTBE, 4×150 mL), washed with brine (200 mL), dried, filtered, and concentrated to give a pale yellow liquid (160 g). ¹H NMR analysis shows ˜15% of dimethyl side product. The pale yellow liquid was acidified with conc. HCl (˜130 mL) and was extracted with ethyl acetate (EtOAc, 3×200 mL). The combined organic layer was washed with brine (200 mL), dried, filtered, and concentrated to give the title compound as a pale yellow liquid (113 g, 72%): ¹H NMR (400 MHz, DMSO-d₆) 3.33 (q, J=6.8 Hz, 1H), 2.10 (s, 3H), 1.31 (d, J=6.8 Hz, 3H).

Step 1b: Alternative Preparation of 2-(methylthio)propanoic acid

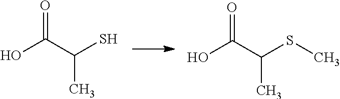

A 250 mL three-necked round bottom flask equipped with an overhead stirrer, a thermocouple, and a reflux condenser was charged with dimethylcarbonate (90 g, 999 mmol) and thiolactic acid (11.4 g, 107 mmol), providing a colorless solution at 19° C. Potassium carbonate (K₂CO₃, 29.9 g, 215 mmol) was added, giving a thick white suspension. Tetrabutylammonium bromide (3.5 g, 10.7 mmol) was added, and the white suspension was heated at 87° C. for 24 h. The reaction mixture was cooled to 50° C., and water (100 mL) was added. The dark yellow solution was cooled in an ice bath, and conc. HCl (35 mL) was added slowly until below pH 5. The mixture was extracted with EtOAc (3×50 mL). The extracts were combined and washed with brine (50 mL). The solvent was concentrated on a rotary evaporator. The mixture was suspended in diethyl ether (50 mL) and gravity filtered to remove the solids. The ether solvent was concentrated by rotary evaporation to give a yellow oil (7.0 g, 58%). ¹H NMR analysis indicated a 3:1 mixture of 2-(methylthio)propanoic acid:methyl 2-(methylthio)propanoate.

Step 1c: Alternative Preparation of 2-(methylthio)propanoic acid

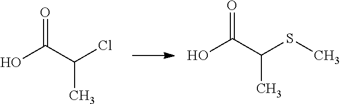

2-Chloropropanoic acid (500 g, 4.61 mol) was added dropwise over a period of 20 to 30 minutes to a stirred solution of 9.5% sodium bicarbonate (NaHCO₃, 4000 mL) at 20-25° C. in a four-neck 20-L round bottom flask. The reaction mixture was stirred for 25 to 30 minutes. DMF (2000 mL) was added, and sodium thiomethoxide (458 g, 6.54 mol) was added in portions at 25-35° C. over a period of 30 to 40 minutes. The reaction mixture was heated at 80-85° C. over a period of 8-10 h. Progress of the reaction was monitored by NMR spectroscopy. After the reaction was complete, the reaction mixture was washed with DCM (2500 mL). The aqueous layer was acidified to pH˜1 with concentrated HCl (˜2.5 volumes) and extracted with MTBE (7×2500 mL). The MTBE layer was concentrated under reduced pressure to 3-4 volumes and was washed with ice cold water. The MTBE layer was dried over anhydrous sodium sulfate and concentrated at 40-45° C. under vacuum to yield the title compound as a colorless liquid (˜380 g, 68%). The ¹H NMR spectral data matched those in Step 1a.

Additional information can be found in Pacey, M. S., et al. J. Antibiot. 1998, 51, 1029-1034; Masya, K., et al. J. Am. Chem. Soc. 1998, 120, 1724-1731; Liu, A., et al. Faming Zhuanli Shenqing, CN 101928271, 29 Dec. 2010; and Wang, X., et al. Faming Zhuanli Shenqing, CN 101928272, 29 Dec. 2010.

Step 2: Preparation of 2-(methyl sulfonyl)propanoic acid (S1a)

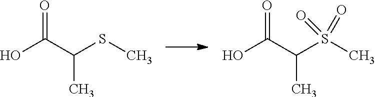

A 5-L round bottom flask with a mechanical stirrer and a temperature probe was charged with 2-(methylthio)propanoic acid (113 g, 1.308 mol) and MeOH (2.5 L). A suspension of OXONE® (Potassium peroxymonosulfate, 593 g, 2.68 mol) in water (1.25 L) was added in portions, maintaining the temperature <45° C. throughout the addition, and the white suspension was stirred for 20 h at room temperature. The solvent (MeOH) was removed, and the aqueous mixture was extracted with EtOAc (4×200 mL). The combined organic extracts were dried, filtered, and concentrated to give a colorless oil (123 g), which was co-evaporated with dichloromethane (DCM). The title compound was isolated as white solid (123 g, 86%): ¹H NMR (400 MHz, DMSO-d₆) 4.32 (q, J=7.2 Hz, 1H), 3.11 (s, 3H), 1.43 (d, J=7.2 Hz, 3H).

Step 3a: Preparation of 2-(methyl sulfonyl)propanoyl chloride (S1b-1)

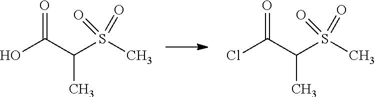

A 1-L round bottom flask equipped with a reflux condenser was charged with 2-(methylsulfonyl)propanoic acid (103 g, 0.68 mol) and thionyl chloride (250 mL, 5 molar equivalents (equiv)), and the suspension was heated to reflux at 80° C. After stirring at reflux for 15 minutes, the suspension became a pale-yellow solution. The solution was heated for another 90 minutes and cooled to room temperature. The thionyl chloride was removed by rotary evaporation. The title compound was isolated as a yellow oil (95 g, 86%): ¹H NMR (400 MHz, DMSO-d₆) 4.32 (q, J=7.2 Hz, 1H), 3.10 (s, 3H), 1.31 (d, J=7.2 Hz, 3H).

Step 3b: Alternative preparation of 2-(methyl sulfonyl)propanoyl chloride (S1b-1)

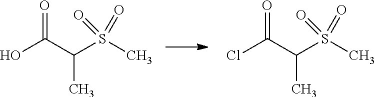

A 100 mL round bottom flask equipped with a reflux condenser was charged with 2-(methylsulfonyl)propanoic acid (5.0 g, 32.9 mmol) and toluene (20 mL, 4 volumes). Thionyl chloride (7.19 mL, 99 mmol) was added to give a suspension. The suspension was heated at 80 ° C. for 3 h to produce a yellow solution. The reaction mixture was cooled to room temperature and concentrated by rotary evaporation. The title compound was isolated as a yellow oil (4.8 g, 86%): ¹H NMR (400 MHz, CDCl₃) δ 4.30 (q, J=7.3 Hz, 1H), 3.08 (s, 3H), 1.81 (d, J=7.2 Hz, 3H); ¹³C NMR (101 MHz, CDCl₃) δ 169.42, 72.91, 39.11, 12.43; GC-MS m/z 135 (M-Cl).

### PROPHETIC EXAMPLE A

Proposed Synthesis of sodium 2-(methylsulfonyl) propanoate
Step 1a: Proposed Synthesis of methyl 2-(methylthio)propanoate

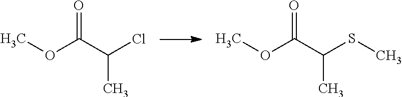

Methyl 2-chloropropionate (1.0 mol) and a phase-transfer catalyst (0.05 mol) are dissolved in toluene (5 volumes). The reaction mixture is cooled to ˜5° C. under nitrogen. A solution of 21% aqueous sodium thiomethoxide (1.0 mol) is added maintaining the temperature around ˜5° C. The reaction is monitored for the formation of product by gas chromatography (GC). If necessary, the reaction mixture is warmed to complete the conversion to product (≥98%). The phases are separated. The organic phase is washed with water. The toluene phase is assayed for weight percent (wt %) of the title compound.

Step 2: Proposed Synthesis of methyl 2-(methylsulfonyl)propanoate

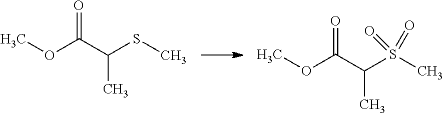

To the solution of methyl 2-(methylthio)propanoate in toluene (Step 1) are added some water, sodium tungstate (0.05 mol) and tetrabutylammonium hydrogen sulfate (0.05 mol). The pH of the reaction mixture is checked, and if necessary, is adjusted to pH 1-2 with a small amount of sulfuric acid. The reaction mixture is cooled to ˜5° C. and an aqueous solution of hydrogen peroxide (30-35%, 1 molar equivalent) is added with cooling to control the temperature at about 5-10° C. Conversion of the reaction is monitored by GC until the ratio of starting sulfide to sulfoxide to sulfone reaches a plateau. The reaction mixture is warmed to ˜60° C. More aqueous hydrogen peroxide (30-35%, 1 to 1.2 molar equivalents) is added to maintain a temperature of about ˜60-65° C. Progress of the reaction is monitored by GC; more hydrogen peroxide may be added to achieve ≥98% conversion to the sulfone. A saturated aqueous sodium bisulfite solution is added in portions until a peroxide test is negative by starch-iodide (KI) paper. The phases are separated, and the aqueous phase is extracted with toluene to recover any product. The toluene phase is concentrated until the wt % of the title compound is ˜20%. The combined toluene phases are assayed for wt % of the title compound.

Step 3: Proposed synthesis of sodium 2-(methylsulfonyl)propanoate

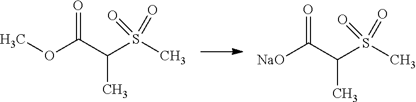

The toluene solution of methyl 2-(methylsulfonyl)propanoate (Step 2 above) is stirred under nitrogen. A 25% aqueous solution of sodium hydroxide (NaOH, 1 molar equivalent) is added to the solution. The reaction mixture is stirred and heated to ˜70° C. Conversion of the reaction is monitored by GC analysis of an aliquot acidified with aqueous hydrochloric acid and extracted into ethyl acetate. When the reaction is ≥98% complete, the solvents (water, methanol, and toluene) are concentrated to ˜50% of the volume. The solids are collected via filtration, washed with toluene, and dried to a minimum moisture content to provide the title compound, which is used in Scheme 2.

### EXAMPLE 2

Preparation of N-(3-chloro-1H-pyrazol-4-yl)-2-(methylsulfonyl)propanamide (S2b)

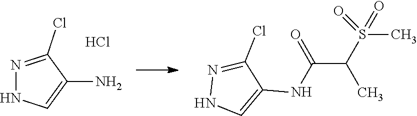

A 250 mL three-neck round bottom flask equipped with a magnetic stirrer, a nitrogen inlet, and a temperature probe was charged with 3-chloro-1H-pyrazol-4-amine hydrochloride (S2a, 4.8 g, 31.2 mmol), tetrahydrofuran (THF; 30 mL) and water (30 mL). NaHCO₃ (10.47 g, 125 mmol) was added in one portion. The reaction mixture was stirred for 5 minutes after completion of addition until off-gassing ceased. The reaction mixture was cooled to ˜5° C. 2-(Methylsulfonyl)propanoyl chloride (S1b-1, 6.26 g, 31.2 mmol) in THF (5 mL) was added by syringe while maintaining a temperature less than 8° C. The reaction mixture was stirred in an ice bath for 2 h. The reaction was neutralized with saturated ammonium chloride and transferred to a separatory funnel. The aqueous layer as extracted with EtOAc (2×). The organic layer was dried over sodium sulfate, filtered and concentrated in vacuo. Residual EtOAc was removed by adding heptane and concentrating in vacuo. The title compound was isolated as an off-white solid (6.79 g, 82%): ¹H NMR (400 MHz, DMSO-d₆) δ 13.01 (s, 1H), 10.08 (s, 1H), 8.09 (s, 1H), 4.34 (q, J=7.0 Hz, 1H), 3.02 (s, 3H), 1.53 (d, J=7.1 Hz, 3H); ESIMS m/z 252 ([M+H]⁺).

### EXAMPLE 3

Preparation of N-(3-chloro-1-(pyridin-3-yl)-1H-pyrazol-4-yl)-2-(methylsulfonyl) propanamide (S3b)

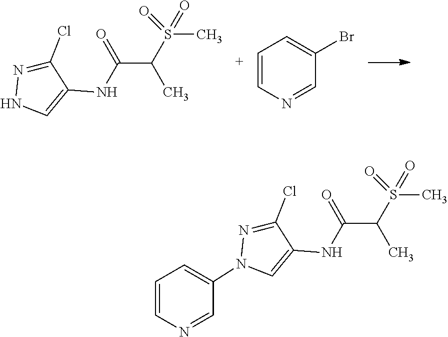 [SMILES: CC(C(=O)Nc1c[nH]nc1Cl)S(C)(=O)=O ; Brc1cccnc1 ; CC(C(=O)Nc1cn(-c2cccnc2)nc1Cl)S(C)(=O)=O]

A 100 mL three-neck round bottom flask equipped with a magnetic stirrer, a temperature probe, a reflux condenser, and a nitrogen inlet was charged sequentially with N-(3-chloro-1H-pyrazol-4-yl)-2-(methylsulfonyl)propanamide (S2b, 4.0 g, 15.89 mmol), acetonitrile (ACN; 20 mL), potassium carbonate (K₂CO₃, 4.39 g, 31.8 mmol), and 3-bromopyridine (1.837 mL, 19.07 mmol). The reaction mixture was sparged with nitrogen for 30 minutes. Copper(I) chloride (0.157 g, 1.589 mmol) and N,N′-dimethylethylenediamine (DMEDA, 0.684 mL, 6.36 mmol) were added. The reaction mixture was sparged with nitrogen for 15 minutes, heated to 75° C., and stirred overnight. Water (20 mL) was added to dissolve solids. Acetonitrile was removed in vacuo, and the pH was adjusted to 5-7 with 2 Normal (N) HCl. Formation of a tan solid was observed. The solid was collected by vacuum filtration and washed with water to afford a tan solid (92% pure by HPLC). The solid was slurried in water (32 mL, 8 volumes) overnight. The solid was collected by vacuum filtration and dried under vacuum to afford the title compound as a light tan solid (4.15 g, 96% purity by HPLC, 76%): ¹H NMR (400 MHz, DMSO-d₆) δ 10.41 (s, 1H), 9.05 (s, 1H), 8.92 (s, 1H), 8.53 (s, 1H), 8.21 (dd, J=2.8, 1.6 Hz, 1H), 7.53 (dd, J=4.4, 0.8 Hz, 1H), 4.45-4.39 (m, 1H), 3.07 (s, 3H) 1.57 (d, J=6.8 Hz, 3H).

The reaction was conducted as in Example 3 in N-methylpyrrolidone (NMP) with a non-isolated yield (HPLC yield based upon an internal standard) of 93%, as well as in N,N-dimethylformamide (DMF) with a non-isolated yield (HPLC yield based upon an internal standard) of 99%.

### EXAMPLE 4

Preparation of N-(3-chloro-1H-pyrazol-4-yl)-2-(methylsulfonyl)propanamide (S2b)
Step 1: Preparation of 2-(methylsulfonyl)propanoyl chloride (S1b-1)

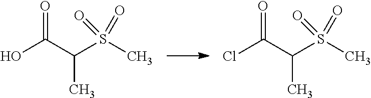

A mixture of 2-(methylsulfonyl)propanoic acid (S1a, 200 g, 1.31 mol) and thionyl chloride (477 mL) was gradually heated to 80° C., and the resulting solution was maintained at 80° C. for 2 hours. After removal of most of the excess thionyl chloride under reduced pressure (rotary evaporation), toluene (50 mL) was added, and the toluene and traces of thionyl chloride were removed under reduced pressure (rotary evaporation) to constant weight (223 g). This was used as is in step 5b (the preparation of S2b).

Step 2: Preparation of N-(3-chloro-1H-pyrazol-4-yl)-2-(methylsulfonyl)propanamide (S2b)

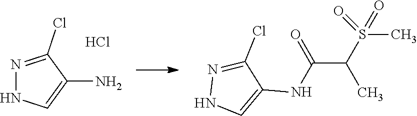

To a mixture of toluene (1.48 L) and water (280 mL) was added 3-chloro-1H-pyrazol-4-amine hydrochloride (S2a, 208 g (184 g on a 100% active ingredient basis, 1.19 mol). The reaction mixture was stirred at 25° C. while a solution of potassium carbonate (198 g in 470 mL of water, 1.43 mol) was added over 20 minutes (with gas evolution). The reaction mixture was cooled and stirred at 0-5° C. while a solution of 2-(methylsulfonyl)propanoyl chloride (S1b-1, 223 g in 370 mL of toluene) was added gradually. After about 2 hours of stirring, the reaction mixture was filtered, and the filter cake was washed sequentially with water (200 mL) and toluene (200 mL). The wetcake was dried in a vacuum oven at 50° C. to constant weight to afford the title compound (S2b, 260 g, assay 98.6 wt % by quantitative NMR analysis; 85.4% yield).

### EXAMPLE 5

Preparation of N-(3-chloro-1H-pyrazol-4-yl)-2-(methylsulfonyl)propanamide (S2b)

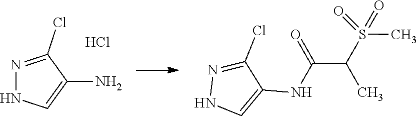

To a stirred solution of THF (74 mL) and water (14 mL) in a 500 mL round bottom flask equipped with a mechanical stirrer and nitrogen inlet 3-chloro-1H-pyrazol-4-amine hydrochloride (S2a, 9.2 g, 60 mmol) was added at 25° C. Triethylamine (12.5 mL, 89.6 mmol) was added over 10 minutes. The reaction mixture was cooled to an internal temperature of 0 to 5° C. 2-(Methylsulfonyl)propanoyl chloride (S1b-1, 11.2 g, 65.7 mmol) dissolved in THF (18 mL) was added to the above mixture. The reaction mixture was stirred for 2 hours and was monitored by HPLC. After complete conversion, the organic layer was separated, and the aqueous layer was extracted with EtOAc (2×30 mL). The combined organic layers were washed with brine solution (50 mL), were dried over anhydrous sodium sulfate, and were concentrated under reduced pressure to get unpurified N-(3-chloro-1H-pyrazol-4-yl)-2-(methylsulfonyl)propanamide (S2b, 12.0 g) as a semi-solid material. The unpurified compound was stirred with 4 volumes of MTBE. The solid was isolated after filtration and drying under vacuum at 50° C. (8.2 g, 55% yield; 96% purity).

### EXAMPLE 6

Preparation of N-(3-chloro-1-(pyridin-3-yl)-1H-pyrazol-4-yl)-2-(methylsulfonyl) propanamide (S3b)

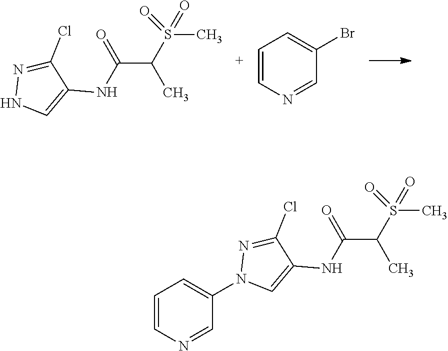 [SMILES: CC(C(=O)Nc1c[nH]nc1Cl)S(C)(=O)=O ; Brc1cccnc1 ; CC(C(=O)Nc1cn(-c2cccnc2)nc1Cl)S(C)(=O)=O]

To a 5-L jacketed reactor with overhead stirrer, nitrogen bubbler, temperature probe, and reflux condenser were charged sequentially, with stirring under nitrogen, acetonitrile (750 mL), N-(3-chloro-1H-pyrazol-4-yl)-2-(methylsulfonyl)propanamide (S2b, 250 g, 0.993 mol), 3-bromopyridine (204 g, 1.29 mol), powdered (325 mesh) anhydrous potassium carbonate (233 g, 1.69 mol), N,N′-dimethylethylenediamine (DMEDA, 35.0 g, 0.397 mol), copper(I) chloride (19.7 g, 0.199 mol), DMSO (75 mL), o-xylene (750 mL), and water (6 g). The reaction mixture was heated at 78-83° C. under nitrogen for 6 hours and then cooled to 60-65° C. A solution of disodium edetate dihydrate (148 g, 0.397 mol) in water (2 L) was added over 45 minutes at 60-65° C.; o-xylene (750 mL) was added over 10 minutes; and the mixture was cooled to 20-25° C. over 40 minutes. Concentrated aqueous HCl (ca. 37%) was added over 20 minutes at 20-25° C. to pH 5.4. The reaction mixture was cooled to 0-5° C. over 80 minutes, was stirred an additional hour at 0-5° C., and was filtered. The filter cake was washed with water (2×500 mL), was suction dried, and was dried in a vacuum oven at 45° C. for 12 hours to afford the title compound S3b (270 g), which was determined by Karl-Fischer titration to contain 5.26 wt % water, indicative of a monohydrate. Yield based on assay of ca. 91 wt %=75%.

Consequently, considering the above the following additional, non-exhaustive, disclosure details (d) are provided.

1d. A molecule, N-(3-chloro-1H-pyrazol-4-yl)-2-(methylsulfonyl)propanamide (S2b), having the following formula

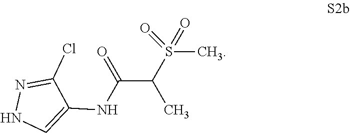

2d. A process for the preparation of a molecule according to 1d the process comprising:
reacting
   (A) 3-chloro-1H-pyrazol-4-amine hydrochloride (S2a)

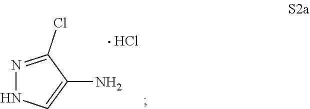

and
(B) an activated carboxylic acid S1b

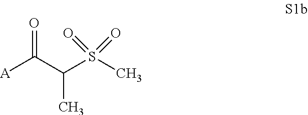

wherein said A of said activated carboxylic acid S1b is selected from the group consisting of Cl, O(C═O)R₁, O(C═O)OR₁, and OR₁, wherein R₁ is (C₁-C₄)alkyl;
in the presence of a base and a solvent.

3d. A process according to 2d wherein said base is an organic base.

4d. A process according to 3d wherein said organic base is selected from pyridine, lutidine, 2-picoline, N,N-diisopropylethylamine, and triethylamine.

5d. A process according to 2d wherein said base is an inorganic base.

6d. A process according to 5d wherein said inorganic base is selected from sodium hydroxide (“NaOH”), potassium hydroxide (“KOH”), potassium carbonate, potassium bicarbonate, sodium carbonate, and sodium bicarbonate.

7d. A process according to any of the previous details 2d through 6d wherein the amount of base is from about 1 mole to about 5 moles of base per mole of S2a.

8d. A process according to any of the previous details 2d through 6d wherein the amount of base is from about 2.0 mole to about 3.5 moles of base per mole of S2a.

9d. A process according to any of the previous details 2d through 8d wherein said solvent is selected from heptanes, chloroform, dichloroethane, toluene, dichloromethane, tetrahydrofuran, 2-methyltetrahydrofuran, methyl tert-butyl ether (“MTBE”), cyclopentyl methyl ether (“CPME”), acetonitrile, ethyl acetate, n-butanol, isopropanol, n-propanol, ethanol, methanol, water, and mixtures thereof.

10d. A process according to any of the previous details 2d through 9d wherein said process is conducted in the presence of an optional amide bond formation catalyst.

11d. A process according to any of the previous details 2d through 10d wherein said process is conducted at a temperature from about −10° C. to about 110° C.

12d. A process according to any of the previous details 2d through 10d wherein said process is conducted at a temperature from about −10° C. to about 90° C.

13d. A process according to any of the previous details 2d through 12d wherein said process is conducted at ambient pressure.

14d. A process according to any of the previous details 2d through 13d wherein the product of said process is isolated.

15d. A process according to any of the previous details 2d through 13d wherein said process is conducted under continuous flow conditions.

16d. A process according to 2d, wherein said activated carboxylic acid S1b is prepared from 2-(methyl sulfonyl)propanoic acid (S1a)

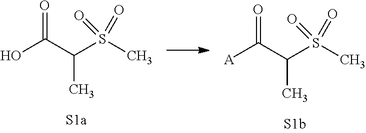

wherein said A of said activated carboxylic acid S1b is selected from the group consisting of Cl, O(C═O)R₁, O(C═O)OR₁, and OR₁, wherein R₁ is (C₁-C₄)alkyl; in the presence of an activator and a solvent.

17d. A process according to 16d wherein said A is Cl.

18d. A process according to 16d wherein said A is O(C═O)R₁, wherein R₁ is (C₁-C₄) alkyl.

19d. A process according to 16d wherein said A is O(C═O)R₁, wherein R₁ is (C₁-C₄) alkyl.

20d. A process according to 16d wherein said A is OR₁, wherein R₁ is (C₁-C₄)alkyl.

21d. A process according to 16d or 17d wherein said activator is oxalyl chloride or thionyl chloride.

22d. A process according to 21d wherein said process is conducted in the presence of an optional acid chloride formation catalyst.

23d. A process according to 16 or 18d wherein said activator is pivaloyl chloride or pivalic anhydride.

24d. A process according to 23d wherein said process is conducted in the presence of an optional mixed anhydride formation catalyst.

25d. A process according to 16d or 19d wherein said activator is methyl chloroformate, ethyl chloroformate, isobutyl chloroformate, or mixtures thereof.

26d. A process according to 25d wherein said process is conducted in the presence of an optional acyl carbonate formation catalyst.

27d. A process according to any of the previous details 16d through 26d wherein the amount of said activator used is about 1.0 moles to about 1.5 moles per mole of 2-(methylsulfonyl) propanoic acid (S1a).

28d. A process according to any of the previous details 16d through 27d wherein said solvent is selected from heptanes, chloroform, dichloroethane, toluene, ethyl acetate, tetrahydrofuran, 2-methyltetrahydrofuran, methyl tert-butyl ether, cyclopentyl methyl ether, dichloromethane, acetonitrile, and mixtures thereof.

29d. A process according to any of the previous details 16d through 27d wherein no solvent is used.

30d. A process according to any of the previous details 16d through 29d wherein said process is conducted at temperatures from about −10° C. to about 110° C.

31d. A process according to any of the previous details 16d through 29d wherein said process is conducted at temperatures from about −10° C. to about 90° C.

32d. A process according to any of the previous details 16d through 31d wherein said process is conducted at ambient pressure.

33d. A process according to any of the previous details 16d through 32d wherein the product of said process is used without isolation from the solvent in a continuous manner in the subsequent reaction.

34d. A process according to any of the previous details 16d through 32d wherein the product of said process is isolated.

35d. A process for the preparation of N-(3-chloro-1-(pyridin-3-yl)-1H-pyrazol-4-yl)-2-(methylsulfonyl) propanamide (S3b)

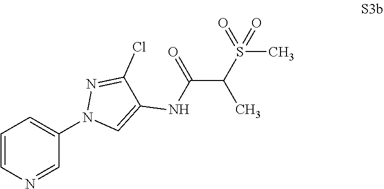 [SMILES: CC(C(=O)Nc1cn(-c2cccnc2)nc1Cl)S(C)(=O)=O]

said process comprising:
reacting
   (A) the molecule according to 1d; and
   (B) a 3-halopyridine S3a

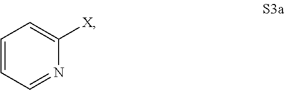

wherein X is Br, Cl, or I;
in the presence of a base, a copper halide, a ligand, and a solvent.

36d. A process according to 35d wherein said base is an organic base.

37d. A process according to 36d wherein said organic base is selected from pyridine, lutidine, 2-picoline, N,N-diisopropylethylamine, and triethylamine.

38d. A process according to 35d wherein said base is an inorganic base.

39d. A process according to 38d wherein said inorganic base is selected from sodium methoxide, sodium ethoxide, lithium hydroxide, sodium hydroxide, potassium hydroxide, cesium hydroxide, calcium hydroxide, sodium diphosphate, potassium phosphate, sodium phosphate, potassium carbonate, potassium bicarbonate, calcium carbonate, cesium carbonate, lithium carbonate, sodium carbonate, and sodium bicarbonate.

40d. A process according to any of the previous details 35d through 39d wherein the amount of base is from about 1 mole to about 5 moles of base per mole of the molecule according to 1d (S2b).

41d. A process according to any of the previous details 35d through 39d wherein the amount of base is from about 2.0 mole to about 3.5 moles of base per mole of the molecule according to 1d (S2b).

42d. A process according to any of the previous details 35d through 41d wherein said copper halide is selected from copper(I) chloride, copper(II) chloride, and copper(I) iodide.

43d. A process according to any of the previous details 35d through 42d wherein said ligand is selected from N,N′-dimethylethylenediamine, triethylenetetramine, bis(2-hydroxyethyl) ethylenediamine, 1-butylimidazole, 8-hydroxyquinoline, L-proline, 2,2-bipyridyl, 1,10-phenanthroline, and pipecolinic acid.

44d. A process according to any of the previous details 35d through 43d wherein said solvent is selected from ethyl acetate, dioxane, tetrahydrofuran, 2-methyltetrahydrofuran, 1,2-dimethoxyethane, dichloromethane, dimethyl sulfoxide, N-methylpyrrolidone, N,N-dimethylformamide, propionitrile, benzonitrile, acetonitrile, xylenes, toluene, water, and mixtures thereof.

45d. A process according to any of the previous details 35d through 44d wherein said process is conducted at temperatures from about 40° C. to about 150° C.

46d. A process according to any of the previous details 35d through 44d wherein said process is conducted at temperatures from about 60° C. to about 120° C.

47d. A process according to any of the previous details 35d through 46d wherein said process is conducted at ambient pressure.

48d. A process according to any of the previous details 35d through 47d wherein the product of said process is isolated.

49d. A process according to any of the previous details 35d through 47d wherein said process is conducted under continuous flow conditions.

50d. A process comprising the steps of:
(A) preparing the molecule according to 1d by reacting
   (i) 3-chloro-1H-pyrazol-4-amine hydrochloride (S2a)

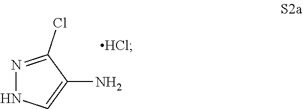

and
(ii) an activated carboxylic acid S1b

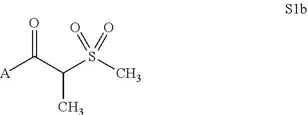

wherein said A of said activated carboxylic acid S1b is selected from the group consisting of Cl, O(C═O)R₁, O(C═O)OR₁, and OR₁, wherein R₁ is (C₁-C₄)alkyl;
in the presence of a base and a solvent; and
(B) preparing N-(3-chloro-1-(pyridin-3-yl)-1H-pyrazol-4-yl)-2-(methylsulfonyl) propanamide (S3b)

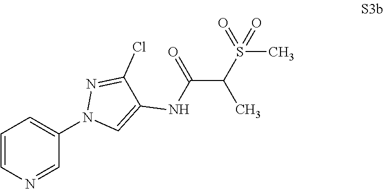 [SMILES: CC(C(=O)Nc1cn(-c2cccnc2)nc1Cl)S(C)(=O)=O]

by reacting
(i) the molecule according to 1d; and
(ii) a 3-halopyridine S3a

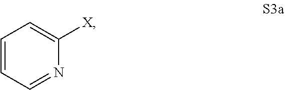

wherein X is Br, Cl, or I;
in the presence of a base, a copper halide, a ligand, and a solvent.

51d. A process according to 50d wherein said process can be modified by any of the previous details 3d to 15d.

52d. A process according to 50d wherein said process can be modified by any of the previous details 36d to 49d.

What is claimed is:

1. A molecule, N-(3-chloro-1H-pyrazol-4-yl)-2-(methylsulfonyl)propanamide (S2b), having the following formula

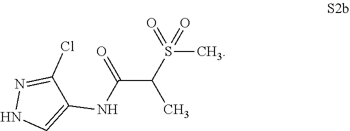

2. A process for the preparation of the molecule according to claim 1 said process comprising:
reacting
(A) 3-chloro-1H-pyrazol-4-amine hydrochloride (S2a) 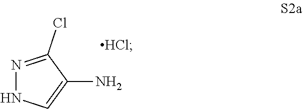
and
(B) an activated carboxylic acid S1b 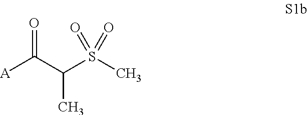
wherein said A of said activated carboxylic acid Sib is selected from the group consisting of Cl, O(C═O)R₁, O(C═O)OR₁, and OR₁, wherein R₁ is (C₁-C₄)alkyl;
in the presence of a base and a solvent.

3. The process according to claim 2 wherein said base is selected from the group consisting of pyridine, lutidine, 2-picoline, N,N-diisopropylethylamine, triethylamine, sodium hydroxide, potassium hydroxide, potassium carbonate, potassium bicarbonate, sodium carbonate, and sodium bicarbonate.

4. The process according to claim 2 wherein said solvent is selected from the group consisting of heptanes, chloroform, dichloroethane, toluene, dichloromethane, tetrahydrofuran, 2-methyltetrahydrofuran, methyl tert-butyl ether, cyclopentyl methyl ether, acetonitrile, ethyl acetate, n-butanol, isopropanol, n-propanol, ethanol, methanol, water, and mixtures thereof.

5. A process for the preparation of N-(3-chloro-1-(pyridin-3-yl)-1H-pyrazol-4-yl)-2-(methylsulfonyl) propanamide (S3b) 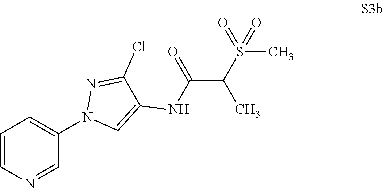 [SMILES: CC(C(=O)Nc1cn(-c2cccnc2)nc1Cl)S(C)(=O)=O]
said process comprising:
reacting
(A) the molecule according to claim 1; and
(B) a 3-halopyridine S3a 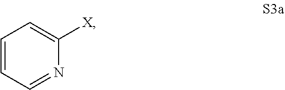
wherein X is Br, Cl, or I;
in the presence of a base, a copper halide, a ligand, and a solvent.

6. The process according to claim 5 wherein said base is selected from the group consisting of pyridine, lutidine, 2-picoline, N,N-diisopropylethylamine, triethylamine, sodium methoxide, sodium ethoxide, lithium hydroxide, sodium hydroxide, potassium hydroxide, cesium hydroxide, calcium hydroxide, sodium diphosphate, potassium phosphate, sodium phosphate, potassium carbonate, potassium bicarbonate, calcium carbonate, cesium carbonate, lithium carbonate, sodium carbonate, and sodium bicarbonate.

7. The process according to claim 5 wherein said copper halide is selected from the group consisting of copper(I) chloride, copper(II) chloride, and copper(I) iodide.

8. The process according to claim 5 wherein said ligand is selected from the group consisting of N,N′-dimethylethylenediamine, triethylenetetramine, bis(2-hydroxyethyl) ethylenediamine, 1-butylimidazole, 8-hydroxyquinoline, L-proline, 2,2-bipyridyl, 1,10-phenanthroline, and pipecolinic acid.

9. The process according to claim 5 wherein said solvent is selected from the group consisting of ethyl acetate, dioxane, tetrahydrofuran, 2-methyltetrahydrofuran, 1,2-dimethoxyethane, dichloromethane, dimethyl sulfoxide, N-methylpyrrolidone, N,N-dimethylformamide, propionitrile, benzonitrile, acetonitrile, xylenes, toluene, water, and mixtures thereof.

10. A process comprising the steps of:
(A) preparing the molecule according to claim 1 by reacting
(i) 3-chloro-1H-pyrazol-4-amine hydrochloride (S2a) 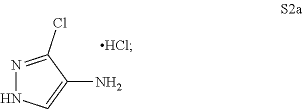
and
(ii) an activated carboxylic acid S1b 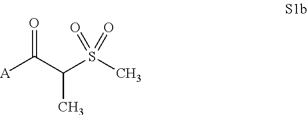
wherein said A of said activated carboxylic acid Sib is selected from the group consisting of Cl, O(C═O)R₁, O(C═O)OR₁, and OR₁, wherein R₁ is (C₁-C₄)alkyl;
in the presence of a base and a solvent; and
(B) preparing N-(3-chloro-1-(pyridin-3-yl)-1H-pyrazol-4-yl)-2-(methylsulfonyl) propanamide (S3b) 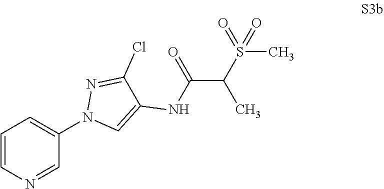 [SMILES: CC(C(=O)Nc1cn(-c2cccnc2)nc1Cl)S(C)(=O)=O]
by reacting
(i) the molecule according to claim 1; and
(ii) a 3-halopyridine S3a 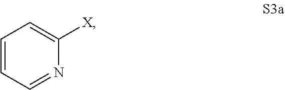
wherein X is Br, Cl, or I;
in the presence of a base, a copper halide, a ligand, and a solvent.

11. The process according to claim 5, wherein said solvent is a mixture of acetonitrile, dimethyl sulfoxide, xylenes, and water.
